SEQUENCE LISTING (2 sequences)

SEQ ID NO: 1 - DNA, 1566 bp, Cyprinus carpio
  CDS (31)..(1530)
gttaacaatttcacacaggaaacagaattcatgagttcgtccaaggaatatatgaatggccacgtgcatctggaggagagtgatatgtatgaatccgatggcaaactgtttctgacggaagcctttaacgttatcatggaagaaattttgcacaaaggtaccgattcggaagagaaagtgtgtgagtggaaagatcccgatcagctgcgtgccctcttggatctggatctgcgtgaacatggggaatcacataaacagctgcttcaacgcgtgcgtgacgttgcacgctattctgtcaaaacctgtcatccgcgcttcttcaatcagctgtttgccggcgtagactatcacgctcttaccggacgcttgctgaccgaaaccctgaatacgagtcagtacacttacgaagtcgctccggtgtttgttcttatggaagaggaagtgctttgcaaattacgcagcctcgtcggatggtctgaaggcgacggtattttctgtcctggcggcagcatgtccaacatgtatgcgatgaatgtggctcgttattgggctttccctcaggttaaaacgcagggtttatgggcaacgccacgtatggcgattttcacctctcaacagggccattacagcatgaagaaagcggcagcctttctgggtattggcacagacaatgtgttcatcgtacaggtcgatgaaagcgggagtatgcgtcctgaggatctcgaagcgaaaatcgtacaagcgaaaagccaggatgccgttcccttctttgtgaacgcaacagccggtacaaccgttcaaggtgcctttgatccgctgaatcgtattgcggacatctcagaacgcaacggcatgtggatgcacgtcgatgcggcttggggtggttcagtgctgtttagcaagaagcacaaacatctggttgcgggcattgaacgtgcaaacagcgtgacttggaatccgcacaaaatgctgctggccgggttacagtgttctgtcgtactgtttcgcgataccacgaatttactgatgcactgccattcggccaaagcgacgtacctcttccaacaagacaagttttacgacacttcgcttgatactggggacaaaagcattcagtgcggtcgcaaggtggattgcttgaaactgtggctgatgtggaaagcgattggcacccatgggttatccgagcgtgtagagaaagcgtttgctttagcacgctatctggtcgaagaaatggaaaagcgcgacaacttcaagctggtgtgcaaaggtccgtttgtaaacgtgtgcttttggtttatcccgccgtcactccgccgcaaagaaaactccgcggattaccaagaacggttgtcgaaagtggcaccggttatcaaagagcggatgatgaaacgtggaacgatgatggtgggttatcagccaatggatggccatgtcaacttctttcgcatggttgttgtcagtccacagttgaccaccaaagacatggacttcttcctggatgagattgaagatctgggcaaagacctgtaagcttggctgttttggcggatgagagaagatatc

SEQ ID NO: 2 - protein, 500 aa, Cyprinus carpio
MSSSKEYMNGHVHLEESDMYESDGKLFLTEAFNVIMEEILHKGTDSEEKVCEWKDPDQLRALLDLDLREHGESHKQLLQRVRDVARYSVKTCHPRFFNQLFAGVDYHALTGRLLTETLNTSQYTYEVAPVFVLMEEEVLCKLRSLVGWSEGDGIFCPGGSMSNMYAMNVARYWAFPQVKTQGLWATPRMAIFTSQQGHYSMKKAAAFLGIGTDNVFIVQVDESGSMRPEDLEAKIVQAKSQDAVPFFVNATAGTTVQGAFDPLNRIADISERNGMWMHVDAAWGGSVLFSKKHKHLVAGIERANSVTWNPHKMLLAGLQCSVVLFRDTTNLLMHCHSAKATYLFQQDKFYDTSLDTGDKSIQCGRKVDCLKLWLMWKAIGTHGLSERVEKAFALARYLVEEMEKRDNFKLVCKGPFVNVCFWFIPPSLRRKENSADYQERLSKVAPVIKERMMKRGTMMVGYQPMDGHVNFFRMVVVSPQLTTKDMDFFLDEIEDLGKDL

(19) United States
(12) Patent Application Publication
(10) Pub. No.: US 2024/0417763 A1
(43) Pub. Date: Dec. 19, 2024

(54) PROCESS FOR PRODUCING TAURINE

(71) Applicant: WACKER CHEMIE AG, Munich (DE)

(72) Inventors: Rupert PFALLER, Munich (DE); Günter WICH, Munich (DE)

(73) Assignee: WACKER CHEMIE AG, Munich (DE)

(21) Appl. No.: 18/712,705

(22) PCT Filed: Nov. 29, 2021
(86) PCT No.: PCT/EP2021/083369

§ 371 Date: May 23, 2024

(51) Int. Cl. C12P 13/04; C12N 9/88

(52) U.S. Cl. CPC C12P 13/04; C12N 9/88; C12Y 401/01011; C12Y 401/01015; C12Y 401/01029; C12Y 402/99

(57) ABSTRACT

A process for producing taurine. Where the taurine is produced from O-acetyl-L-serine (OAS) using biotransformation. In a first processing step (biotransformation 1), L-cysteic acid is produced from OAS using an enzyme selected from a class of OAS sulfhydrylases (EC 4.2.99.8) in the presence of a salt of sulfurous acid. Where the biotransformation is carried out under active pH control. In a second processing step (biotransformation 2), L-cysteic acid is decarboxylated to taurine. Where the OAS concentration in the batch is at least 10 g/L and the OAS sulfhydrylase is CysM.

The invention relates to a process for producing taurine from O-acetyl-L-serine (OAS) by means of biotransformation, wherein in a 1st process step (biotransformation 1), L-cysteic acid is produced from O-acetyl-L-serine (OAS) using an enzyme selected from the class of OAS sulfhydrylases (EC 4.2.99.8) in the presence of a salt of sulfurous acid, and then in a 2nd process step (biotransformation 2), L-cysteic acid is decarboxylated to taurine. Taurine is provided as a result of this biotransformation.

Taurine (2-aminoethanesulfonic acid, CAS number 107-35-7) is an aminosulfonic acid that occurs naturally in nature as a breakdown product of the amino acids cysteine and methionine. Taurine is a constituent of energy drinks and is also used in pet food, for example for cats, or in fish farming (Salze and Davis (2015) Aquaculture 437: 215-229). Taurine is however considered to have health-promoting effects too (Ripps and Shen (2012), Molecular Vision 18: 2673-2686).

In nature, taurine occurs almost exclusively in the animal kingdom, there being only a few examples where it occurs in bacteria, algae or plants. There are various biosynthetic pathways to taurine (for an overview, see for example the KEGG Pathway Database: “Taurine and hypotaurine metabolism”), starting inter alia from L-cysteine. The most important synthetic steps leading from L-cysteine to taurine are shown in equations (1) to (5).
(1) L-Cysteine+O₂→L-Cysteine sulfinic acid
(2) L-Cysteine sulfinic acid+½O₂→L-Cysteic acid
(3) L-Cysteine sulfinic acid→Hypotaurine+CO₂
(4) Hypotaurine+½O₂→Taurine
(5) L-Cysteic acid→Taurine+CO₂
(1) In a first step, L-cysteine is oxidized by the enzyme cysteine dioxygenase (CDO, EC 1.13.11.20) to L-cysteine sulfinic acid (3-sulfinoalanine, CAS number 207121-48-0).
(2) Cysteine sulfinate oxidase (previously a rather hypothetical enzymatic step) further oxidizes L-cysteine sulfinic acid to L-cysteic acid ((R)-2-amino-3-sulfopropionic acid, CAS number 23537-25-9).
(3) Cysteine sulfinic acid decarboxylase (CSAD, EC 4.1.1.29) decarboxylates L-cysteine sulfinic acid to hypotaurine (2-aminoethanesulfinic acid, CAS number 300-84-5).
(4) Hypotaurine is oxidized to taurine, previously not fully explained.
(5) In a step similar to (3), L-cysteic acid is decarboxylated to taurine by suitable CSAD enzymes.

Taurine for commercial use is currently produced chemically. One known process is for example that of Changshu Yudong Chemical Factory, which starts with ethylene and leads to taurine via ethyleneimine. With the consumer-driven trend away from chemically produced ingredients to sustainably produced ingredients, biotechnological processes for the production of taurine are increasingly being investigated. The prior art relies on metabolic engineering approaches in which suitable biosynthesis genes are expressed heterologously in a production strain and produce taurine or its biosynthetic precursor hypotaurine.

Honjoh et al. (2010), Amino Acids 38: 173-1183 describe a genetically engineered yeast strain heterologously expressing the CDO gene and CSAD gene from carp (Cyprinus carpio). When L-cysteine was added to growth of the genetically engineered strain, what was observed was the production of hypotaurine as the main product and additionally a relatively low proportion of taurine. Treatment with H₂O₂ resulted in oxidation of hypotaurine to taurine. Even though S. cerevisiae itself is capable of producing cysteine from its own metabolism, the production of hypotaurine and taurine required the external addition of L-cysteine to the growth medium.

Tevatia et al. (2019), Algal Research 40: 101491, likewise use heterologously expressed CDO and CSAD genes from carp for production of taurine in the alga Chlamydomonas reinhardtii. The intracellular taurine yield was 0.14 mg of taurine per g of dry biomass.

Joo et al. (2018), J. Agric. Food Chem. 66: 13454-13463, describe a genetically engineered strain of the bacterium Corynebacterium glutamicum with an output of 0.5 g/L taurine when grown in a shake flask. It was apparent that taurine was accumulated intracellularly and not secreted into the growth medium. The synthesis of taurine was achieved by heterologously expressing in the strain the genes of an L-cysteic acid synthase, a cysteine dioxygenase and an L-cysteine sulfinic acid decarboxylase. In addition, a repressor gene in relation to methionine and cysteine biosynthesis was inactivated, which simultaneously achieved improved sulfur uptake. Besides the low yield, the fact that taurine is produced intracellularly can be considered to be a fundamental disadvantage in view of product isolation. This requires complex disruption of the cells in order to release the taurine for further processing.

WO17213142A1 (Ajinomoto) describes a taurine-producing strain obtained by heterologous expression of a cysteine dioxygenase and an L-cysteine sulfinic acid decarboxylase in a strain capable of cysteine production. The main product was hypotaurine with a maximum yield of 450 μM, which could be converted into taurine only through subsequent treatment with alkali and only in low yields.

U.S. Pat. No. 9,267,148B2, US2012/0222148A1, US2018/0028474A1, US2019/0085339, WO2017/176277A1 and WO2019/094051 (Plant Sensory Systems) describe taurine-producing strains and organisms comprising in various forms the heterologous expression of a cysteine dioxygenase and an L-cysteine sulfinic acid decarboxylase. The main focus of the applications is the production of taurine in plants, though nothing is said about yields.

US20190062757A1 (KnipBio) describes heterologous production strains for production of taurine or its precursors.

The prior art thus discloses various metabolic engineering approaches for producing hypotaurine or taurine in heterologous production systems. Where product yields are disclosed by the metabolic engineering approaches, they are too low for industrial use.

The prior art also provides processes for producing non-proteinogenic (unnatural) amino acids, for example by direct fermentation of microorganisms (EP 1 191 106 B1, Wacker) or biotransformation of OAS catalyzed by O-acetyl-L-serine sulfhydrylase (OAS sulfhydrylase) (EP 1 247 869 B1, Wacker). The processes are based on an OAS sulfhydrylase catalyzing the reaction of OAS with a nucleophile to form a non-proteinogenic amino acid according to the general equation (6):

OAS+Nucleophile→Unnat. amino acid+Acetate (6)

In EP 1 247 869 B1 (Wacker), a multitude of different nucleophiles were tested for their suitability as nucleophile for the reaction with OAS catalyzed by an OAS sulfhydrylase, including selenides, selenol, azides, cyanides, azoles and isoxazolinones. In addition, sulfur compounds from the group of thiosulfates and thiols of the general formula H—S—R were also used, the radical R being a monovalent substituted or unsubstituted alkyl, alkoxy, aryl or heteroaryl radical. These processes are unsuitable for producing L-cysteic acid and/or taurine.

It is an object of the present invention to provide a biotechnological process for producing taurine by biotransformations by bypassing a heterologous production strain generated by metabolic engineering.

The object is achieved by a process for producing taurine from O-acetyl-L-serine (OAS) by means of biotransformation, wherein in a 1st process step (biotransformation 1), L-cysteic acid is produced from O-acetyl-L-serine (OAS) using an enzyme selected from the class of OAS sulfhydrylases (EC 4.2.99.8) in the presence of a salt of sulfurous acid, and then in a 2nd process step (biotransformation 2), L-cysteic acid is decarboxylated to taurine.

As disclosed in the accompanying application Co12102 and the examples of the present invention, it has been found that, surprisingly, salts of sulfurous acid (referred to hereinafter as sulfites or SO₃²⁻) are suitable as nucleophile in reaction (6) and, in an extension of EP 1 247 869 B1 (Wacker), allow the synthesis of the non-proteinogenic amino acid L-cysteic acid (biotransformation 1) in a previously unknown reaction according to equation (7).

OAS+SO₃²⁻→L-Cysteic acid+Acetate (7)

Equally unexpectedly, the L-cysteic acid produced in biotransformation 1 could be decarboxylated to taurine without further workup according to equation (5) by a recombinantly produced CSAD enzyme. (Biotransformation 2).

The advantage of the process according to the invention for producing taurine is that it is a sustainable and technically feasible economically viable biotransformation process for producing taurine. Environmentally hazardous chemicals can be dispensed with. Fossil raw materials are not consumed and toxic chemical waste and/or waste gases are not produced. The production process of the present invention is therefore environmentally friendly and sustainable. Moreover, the process does not require either extreme reaction conditions or special equipment and is therefore easy to technically implement. There is increasing demand for such processes.

Taurine from the process according to the invention can be either used further directly without further workup steps or enriched by means of known methods.

In the context of the present invention, production processes are distinguished as follows:
1. Chemical processes
2. Biotechnological processes:
   a) by metabolic engineering
      Metabolic engineering (also called “pathway design”), in contrast to biotransformation, is a biotechnology method in which metabolic pathways of an organism are modified by optimization or modification of genetic and regulatory processes. New or modified enzymes can be introduced into an organism by supplementation of the genome with genes of enzymes, or genes of endogenous enzymes can be expressed at an enhanced or attenuated level, thereby establishing new metabolic pathways in an organism or enhancing or attenuating existing metabolic pathways. The goal of metabolic engineering is for the organism to produce either a new metabolite or a cell-endogenous metabolite at increased yield. A metabolic engineering process does not use starting materials specific for the metabolite, such as an enzyme substrate, for example OAS in the present invention; instead, it only uses a nutrient medium, also referred to as growth medium, which is required for the growth of the organism in question and is composed of a carbon source (e.g., glucose), a nitrogen source (e.g., an ammonium salt or a complex amino acid mixture such as peptone or yeast extract) and other salts required for growth. Such nutrient media are known to a person skilled in the art from microbiological practice.
   b) by biotransformation
      Biotransformation is defined as the transformation of one or more reactants into a product under enzymatic catalysis, the enzyme substrate being added with the enzyme to a reaction batch. In the reaction batch, the enzyme substrate added, such as OAS or L-cysteic acid in the present invention, is converted enzymatically. In the present invention, this is accomplished for OAS by an enzyme selected from the class of OAS sulfhydrylases (EC 4.2.99.8) in the presence of a salt of sulfurous acid) according to equation (7) and for L-cysteic acid by an enzyme selected from the class of cysteine sulfinic acid decarboxylases (CSAD, EC 4.1.1.29) according to equation (5). The reactant(s) can stem from chemical or biotechnological production. The OAS used in the process according to the invention can stem, for example, from chemical synthesis or from biotechnological production by fermentation of a production strain. The L-cysteic acid used in the process according to the invention can stem, for example, from chemical synthesis or from biotechnological production by biotransformation of OAS. The enzyme(s) used for enzymatic catalysis stem from biotechnological production by growth of production strains, for example by fermentation, or biological material containing the enzyme(s) is used (e.g., plants, fungi, algae, animal organs). The biomass from the growth of the production strain, or the biological material, can be used directly, or the enzyme is isolated therefrom depending on the requirements of the biotransformation. The enzymes CysM and CSADcc used in the process according to the invention stem from biotechnological production by fermentation of a production strain.

In the context of the present invention, a reaction batch is defined as a mixture of reactant (starting material), enzyme and optionally other reactants, in which the reactant is converted into a product.

The yield of the reaction within the meaning of the invention is defined as the amount of reactant used that is converted into the product under reaction conditions. The yield can be expressed in terms of absolute amount (g or mmol), as a volume yield in terms of absolute amount of product per unit volume (mM or g/L) or as a relative yield of product as a percentage of reactant used (taking into account the molecular weights of the reactant and of the product), also referred to as percent yield.

Fermentation is a process step for the production (cultivation) of cell cultures on an industrial scale, in which a preferably microbial production strain is made to grow under defined conditions of culture medium, temperature, pH, oxygen supply, and mixing of the medium. Depending on the configuration (genetic makeup) of the production strain, the aim of fermentation is to produce a protein/enzyme or a metabolite, in each case in the highest possible yield for further use. The components of the process according to the invention, OAS, OAS sulfhydrylase and cysteine sulfinic acid decarboxylase, can be produced by fermentation. The final product of fermentation is a fermenter broth consisting of the biomass of the cells of the production strain (fermenter cells) and the fermentation medium (fermentation supernatant) which has been removed from the biomass and which has formed during fermentation from the growth medium and the metabolites secreted by the fermenter cells. The target products of fermentation can be present in the fermenter cells or in the fermentation medium. For instance, OAS is found in the fermentation medium, whereas the enzymes OAS sulfhydrylase and CSADcc are found in the fermenter cells.

Open reading frame (ORF, synonymous with cds or coding sequence) refers to a region of DNA or RNA that begins with a start codon and ends with a stop codon and encodes the amino acid sequence of a protein. The ORF is also referred to as the coding region or structural gene.

Gene refers to the section of DNA that contains all the basic information for producing a biologically active RNA. A gene contains the section of DNA from which a single-stranded RNA copy is produced by transcription and also the expression signals involved in the regulation of this copying process. The expression signals include for example at least one promoter, a transcription start, a translation start, and a ribosome binding site (RBS). A terminator and one or more operators are additional possible expression signals.

An mRNA, also known as messenger RNA, is a single-stranded ribonucleic acid (RNA) that carries the genetic information for the synthesis of a protein. An mRNA provides the assembly instructions for a particular protein in a cell. The mRNA molecule conveys the requisite message for protein synthesis from the genetic information (DNA) to the ribosomes responsible for protein synthesis. In a cell it is formed as a transcript of a section of DNA corresponding to a gene. The genetic information stored in the DNA is unchanged by this process.

Genes of eukaryotic organisms are predominantly what are known as mosaic genes and, unlike prokaryotic genes, also contain non-coding sections known as introns (intragenic regipns). Coding sequences, which are known as exons (xpressed regions), are sections of DNA of a eukaryotic gene that, after being transcribed into RNA, are translated by the ribosomes into the amino acid sequence of a protein. After transcription of DNA into RNA, the introns are spliced from the primary transcript. The protein-coding RNA with introns removed is referred to as messenger RNA (mRNA) or “mature” mRNA. This undergoes further modifications such as capping and polyadenylation. The coding region of the mature mRNA is then translated into the protein sequence. If a eukaryotic gene containing an exon/intron structure is to be expressed in prokaryotic organisms, it is necessary to back-translate the protein sequence or coding region of the mature mRNA into intron-free DNA, since no processing of the exon/intron structure takes place in prokaryotes. When referring in the context of this invention to gene sequences derived from the protein sequence or gene sequences derived from mRNA, what is meant is precisely this process of back-translation. It is preferable that sequence optimization, i.e. adaptation to the codon usage of the corresponding prokaryote (codon optimization), takes place concomitantly with the back-translation of the protein sequence or the mRNA sequence into a DNA sequence.

A gene construct refers to a DNA molecule in which a gene is linked to other genetic elements (e.g., promoter, terminator, selection marker, origin of replication). A gene construct in the context of the invention is a circular DNA molecule and is referred to as a plasmid, vector or expression vector. The genetic elements of the gene construct give rise to the extrachromosomal inheritance thereof during cell growth and to the production of the protein encoded by the gene.

The abbreviation WT (Wt) refers to the wild type. Wild-type gene refers to the form of the gene that arose naturally through evolution and is present in the wild-type genome. The DNA sequence of Wt genes is publicly available in databases such as NCBI.

### Biotransformation 1:

The process according to the invention for producing taurine requires the availability of OAS. Conceivable are chemical processes for producing OAS, for example by acetylation of L-serine, which is costly because of the high prices of L-serine, or else the production of the racemate O-acetyl-D/L-serine, which can be used directly, or OAS is obtained from the racemate beforehand, for example by resolution. In the case of direct acetylation, N-acetyl-L-serine (NAS) may be formed as a by-product, for example by nonselective acetylation on the hydroxyl or amino group of L-serine or the known rearrangement of OAS to NAS at neutral to alkaline pH values (Tai et al. (1995), Biochemistry 34: 12311-12322), which reduces yields or requires the prior introduction of a protective group on the amino group of L-serine. The direct acetylation of L-serine is therefore not practical for an economically viable process.

The biotechnological production of OAS is known. This involves the use of organisms which exhibit deregulated cysteine metabolism and therefore provide a high level of OAS. A fermentative process for producing OAS is disclosed in EP 1 233 067 B1 (Wacker) and described in example 1 of the present invention.

Preference is given to the biotechnological production of OAS, especially preferably using the Escherichia coli (E. coli) strain W3110/pACYC-cysEX-GAPDH-ORF306 (example 1), deposited in accordance with the Budapest Treaty at the DSMZ-German Collection of Microorganisms and Cell Cultures GmbH (Braunschweig) under the number DSM 13495.

An advantage of the present invention is that an OAS-containing fermenter broth, as obtained for example from a fermentation carried out according to EP 1 233 067, can be used directly in the process according to the invention as a source of OAS, as a fermentation supernatant following removal of the particulate biomass, for example by centrifugation without further workup, purification or isolation steps, including extraction, adsorption, ion exchange chromatography, precipitation or crystallization. This procedure is particularly economical and avoids isolating an unstable compound.

A person skilled in the art can use isotope analysis to determine whether a substance they wish to use in the process, such as OAS or L-cysteic acid, stems from chemical or fermentative production. An isotope analysis method capable of differentiating is described for example in Sieper et al., Rapid Commun. Mass Spectrom. (2006) 20: 2521-2527 and is based on determination of the isotope ratios for e.g. carbon or nitrogen, which vary according to whether a product stems from chemical (petroleum-based) production or fermentative production (from plant-based raw materials).

OAS sulfhydrylases have hitherto been isolated from various plants and microorganisms. In E. coli, there are for example two OAS sulfhydrylase enzymes, which are referred to as CysK and CysM. The associated genes are likewise known and are designated cysK and cysM, respectively.

OAS sulfhydrylases within the meaning of the present invention are characterized in that they can catalyze the synthesis of the proteinogenic amino acid L-cysteine from OAS according to equation (8), the nucleophile used in this case being sulfide. Both CysM-related and CysK-related enzymes are therefore OAS sulfhydrylases within the meaning of the present invention.

OAS+S²⁻→L-Cysteine+Acetate (8)

Although both enzymes have a very similar reaction mechanism and are involved in the biosynthesis of L-cysteine, CysM, unlike CysK, has a variable substrate spectrum with regard to the nucleophile which can react with OAS according to equation (6). For example, it is known that CysM, unlike CysK, is capable of catalyzing reaction of OAS with thiosulfate to form S-sulfocysteine (CAS number 1637-71-4). This reaction plays an important role in bacterial growth with thiosulfate as the only source of sulfur. Furthermore, EP 1 247 869 B1 (Wacker) discloses the use of CysM for production of non-proteinogenic amino acids.

Preferably, the process is characterized in that the OAS sulfhydrylase is a bacterial enzyme, particularly preferably CysM and especially preferably CysM from the strain E. coli.

The process is preferably characterized in that the OAS sulfhydrylase, including preferably CysM, stems from fermentative production, particularly preferably from the fermentation of an E. coli strain and especially preferably from the fermentation of the strain E. coli DH5α/pFL145.

Example 2 discloses a procedure for the fermentative biotechnological production of CysM with the strain E. coli DH5α/pFL145. The production strain consists of a host strain, such as E. coli DH5α in this case, and a gene construct suitable for expressing the OAS sulfhydrylase, preferably the gene construct pFL145. Host strain and gene construct, and the production of the production strain, are described in EP 1 247 869 B1. The production strain is deposited according to the Budapest Treaty at the DSMZ-German Collection of Microorganisms and Cell Cultures GmbH (Braunschweig) under the number DSM 14088.

The fermenter broth obtained by fermentation of the production strain consists of the fermenter cells containing the OAS sulfhydrylase and of the fermentation medium (fermentation supernatant). In the process according to the invention, either the fermenter broth without further workup can be used, or else a suspension of the fermenter cells (resuspended fermenter cells), for example in a buffer, can be used after isolation thereof from the fermenter broth, for example by centrifugation or filtration. Example 2 describes the production of resuspended CysM-containing fermenter cells of the strain E. coli DH5α/pFL145.

It is furthermore conceivable that the OAS sulfhydrylase is used in the form of a cell homogenate after mechanical disruption of the fermenter cells or in the form of chemically permeabilized cells (e.g., by chloroform) or else as a cell extract after removal of particulate constituents from the cell homogenate or else as an enzyme purified by chromatography, for example. Preference is given to the use of the OAS sulfhydrylase as a fermenter broth without further workup, as a cell suspension of resuspended fermenter cells or else as a cell homogenate after mechanical disruption of resuspended fermenter cells or in the form of chemically permeabilized cells (e.g., by chloroform). Particular preference is given to the use of the OAS sulfhydrylase in the form of resuspended fermenter cells or as a cell homogenate. Especial preference is given to the use of resuspended fermenter cells of the OAS sulfhydrylase production strain.

In the process according to the invention, OAS reacts with sulfite to form L-cysteic acid under catalysis by an OAS sulfhydrylase according to equation (7).

Sulfurous acid forms a multitude of chemical species simultaneously present in reversible equilibria, the respective suitability of which as nucleophile in the biotransformation according to the invention was not foreseeable. It is thus known that sulfurous acid (H₂SO₃) is the aqueous solution of gaseous SO₂ and is, as a dibasic acid, present in different equilibria depending on the pH of the aqueous solution, the species of said equilibria also varying in suitability as nucleophile. The following equilibria (9) to (13) are known:

SO₂(gaseous)<->SO₂(dissolved) (9)

SO₂(dissolved)+H₂O<->H₂SO₃ (10)

H₂SO₃<->HSO₃⁻+H⁺ (11)

HSO₃⁻<->SO₃²⁻+H⁺ (12)

2HSO₃⁻<->S₂O₅²⁻+H₂O (13)

Sulfurous acid and its salts are used as preservatives in the food industry, since they exhibit an antimicrobial effect. This means that sulfurous acid and its salts can kill microorganisms, which is attributable to the inactivation of the enzymes necessary for microorganism viability. A person skilled in the art would therefore expect that the CysM enzyme would also be inactivated when using sulfurous acid or its salts and that L-cysteic acid would not be preparable by the process disclosed in EP 1 247 869 B1.

For the aforementioned reasons, it was surprising to a person skilled in the art that, when sulfite and OAS are used in a biotransformation, L-cysteic acid can be produced as the starting compound for a biotechnological process for producing taurine.

In principle, all conceivable salts of sulfurous acid are suitable for the reaction. Preferably, the process is characterized in that the salt of a sulfurous acid used is Na₂SO₃, K₂SO₃, (NH₄)₂SO₃, NaHSO₃ (or its anhydride Na₂S₂O₅) or KHSO₃. Particularly preferably, the salt of a sulfurous acid used is Na₂SO₃, NaHSO₃ (or its anhydride Na₂S₂O₅) or (NH₄)₂SO₃ and especially preferably Na₂SO₃ or NaHSO₃ (or its anhydride Na₂S₂O₅).

It is conceivable to use gaseous sulfur dioxide, the anhydride of sulfurous acid, which can be introduced into the reaction batch, where it is hydrated to sulfurous acid H₂SO₃ and, depending on the pH, is in an equilibrium with the deprotonated forms HSO₃⁻ and SO₃²⁻.

Preferably, the process is characterized in that the concentration of the salt of sulfurous acid is at least in equimolar concentration to OAS. Particularly preferably, the salt of sulfurous acid is at least in 1.5-fold molar excess, especially preferably in at least five-fold molar excess to OAS.

In a particularly preferred embodiment, the process for producing taurine is characterized in that both the OAS sulfhydrylase and OAS are produced by fermentation.

OAS as the reactant of the biotransformation process according to the invention isomerizes from a pH of approx. pH 7 to N-acetyl-L-serine (NAS) and is then no longer suitable for reaction with sulfite to form L-cysteic acid. The mechanism of the reaction was studied in Tai et al. (1995), Biochemistry 34: 12311-12322 and involves an intramolecular, nucleophilic attack by the deprotonated amino group on the carbonyl carbon of the acyl radical. This reaction is suppressed with decreasing pH, and so the compound is stable at pH 4.0, for example.

The biotransformation process according to the invention is therefore preferably distinguished by the fact that the reaction is carried out under pH conditions which minimize the isomerization of OAS to NAS.

The reaction temperature of biotransformation 1 is preferably chosen between 5° C. and 70° C. Preference is given to a reaction temperature between 10° C. and 60° C., particular preference to between 15° C. and 50° C., and especial preference to between 20° C. and 40° C.

The OAS concentration in the batch is preferably at least 1 g/L, particularly preferably at least 10 g/L, and especially preferably at least 40 g/L.

In the biotransformation of OAS, the molar yield of L-cysteic acid based on the molar amount of OAS used is preferably at least 60%, particularly preferably at least 70% and especially preferably at least 80%.

In a further preferred embodiment of biotransformation process 1, the substrate OAS is metered into the reaction batch composed of OAS sulfhydrylase and sulfite from a reservoir in a so-called feed process (example 5). Isomerization of OAS to NAS in the OAS-containing reservoir is avoided by preferably setting a pH of 56.5, particularly preferably a pH of <6.0 and especially preferably a pH of 5.5. At the same time, the pH in the reaction batch is adjusted in such a way that it promotes the reaction to form L-cysteic acid, preferably pH 57.5, particularly preferably pH 57.0 and especially preferably pH 56.5.

According to equation (7), the reaction of OAS to form L-cysteic acid releases stoichiometric amounts of acetic acid, which may lead to a decrease in pH in the batch as the reaction proceeds. Since an excessively low pH affects the activity of the OAS sulfhydrylase, preference should be given to preventing an excessively large drop in pH. This can be done passively by a suitable highly concentrated buffer in the batch or can be achieved actively by a measurement and control unit. Particular preference is given to active pH control by a measurement and control unit, as disclosed in example 5, which, in the event of deviation of the pH from the target value, restores the desired pH by metered addition of an alkaline solution or acid (so-called pH-stat method).

Biotransformation 1 can be carried out in discontinuous or continuous operation. In discontinuous operation (batch operation), all reactants are added to the batch in the course of the reaction and the batch is worked up after the reaction has ended. In continuous operation, OAS, CysM enzyme and a salt of sulfurous acid are constantly metered in during the reaction and a solution containing the product L-cysteic acid is simultaneously removed from the batch. What is established is a steady state in which the reactants are metered in in such a way that they can completely react to form the product L-cysteic acid during the residence time in the reaction vessel. A process for continuous production of unnatural amino acids is disclosed in, for example, EP 1 247 869 B1 (Wacker). Biotransformation 1 is preferably a discontinuous process.

### Biotransformation 2:

The process for producing taurine comprises the production of L-cysteic acid in biotransformation 1 followed by the decarboxylation of L-cysteic acid to taurine in biotransformation 2.

The decarboxylation of L-cysteic acid to taurine can be effected chemically or under enzymatic catalysis in a biotransformation. Thermal decarboxylation at high temperatures under metal catalysis, which is not considered sustainable, is known, but it has the disadvantage of being energy-intensive and producing a high proportion of by-products.

Preferably, the process is characterized in that L-cysteic acid is decarboxylated to taurine using an enzyme from the class of L-cysteine sulfinic acid decarboxylases (CSAD, EC 4.1.1.29), aspartate 1-decarboxylases (EC 4.1.1.11) or glutamate decarboxylases (EC 4.1.1.15).

Particular preference is given to enzymes from the class of L-cysteine sulfinic acid decarboxylases (CSAD, EC 4.1.1.29) for the enzymatically catalyzed decarboxylation of L-cysteic acid to taurine. CSAD enzymes are known to decarboxylate L-cysteine sulfinic acid to hypotaurine according to equation (3). To varying degrees, these enzymes are also capable of decarboxylating L-cysteic acid as substrate to taurine according to equation (5). As disclosed in examples 7 to 11, the CSADcc enzyme from Cyprinus carpio (carp), for example, is suitable for decarboxylating L-cysteic acid to taurine.

Enzymes of the class EC 4.1.1.29 (CSAD) are mainly found in Metazoa (multicellular animals), including mammals. Enzymes having CSAD activity can also be found in single-cell organisms, such as in algae, for example from the genus Synechoccocus, and also in bacteria or fungi. Preferably, the process according to the invention for producing taurine is characterized in that use is made of CSAD enzymes of the class EC 4.1.1.29 from mammals, selected from human (Homo sapiens), cattle (Bos taurus), rat (Rattus norvegicus) or mouse (Mus musculus), and from fishes such as carp (Cyprinus carpio), particularly preferably enzymes from human (Homo sapiens), rat (Rattus norvegicus) or carp (Cyprinus carpio).

Especial preference is given to the CSAD enzyme from carp (CSADcc, Cyprinus carpio). The examples use a CSADcc enzyme, derived from the protein sequence of the Wt CSAD enzyme from Cyprinus carpio, having a DNA sequence of the cds as disclosed in SEQ ID NO: 1, nt 31-1530, and referred to as CSADcc cds, encoding a protein having the amino acid sequence in SEQ ID NO: 2 and referred to as CSADcc.

The DNA sequence forming the basis of the Wt CSADcc amino acid sequence is available in the NCBI (National Center for Biotechnology Information) database under the GenBank sequence ID: AB220585.1 (cds: nt 82-1584). Derived from the corresponding Wt CSADcc amino acid sequence was a CSADcc cds DNA sequence (SEQ ID NO: 1, nt 31-1530) codon-optimized for expression in E. coli and encoding an identical amino acid sequence. Publicly available software programs are available for codon optimization, such as the Eurofins Genomics GENEius software used in example 6. The DNA from SEQ ID NO: 1 was produced synthetically in a known manner, as provided by contractors such as Eurofins Genomics.

Especially preferably, the L-cysteine sulfinic acid decarboxylase is SEQ ID NO: 2 or a sequence homologous to this sequence.

Homologous sequences are to be understood as meaning that the DNA or amino acid sequences are at least 80%, preferably at least 90% and particularly preferably at least 95% identical, each alteration in the homologous sequence being selected from insertion, addition, deletion, and substitution of one or more nucleotides or amino acids.

The degree of DNA identity is determined by the “nucleotide blast” program, which can be found at http://blast.ncbi.nim.nih.gov/ and is based on the blastn algorithm. The algorithm parameters used to align two or more nucleotide sequences were the default parameters. The default general parameters are: Max target sequences=100; Short queries=“Automatically adjust parameters for short input sequences”; Expect Threshold=10; Word size=28; Automatically adjust parameters for short input sequences=0. The corresponding default scoring parameters are: Match/Mismatch Scores=1, −2; Gap Costs=Linear.

Protein sequences are compared using the “protein blast” program at http://blast.ncbi.nlm.nih.qov/. This program uses the blastp algorithm. The algorithm parameters used to align two or more protein sequences were the default parameters. The default general parameters are: Max target sequences=100; Short queries=“Automatically adjust parameters for short input sequences”; Expect Threshold=10; Word size=3; Automatically adjust parameters for short input sequences=0. The default scoring parameters are: Matrix=BLOSUM62; Gap Costs=Existence: 11 Extension: 1; Compositional adjustments=Conditional compositional score template adjustment.

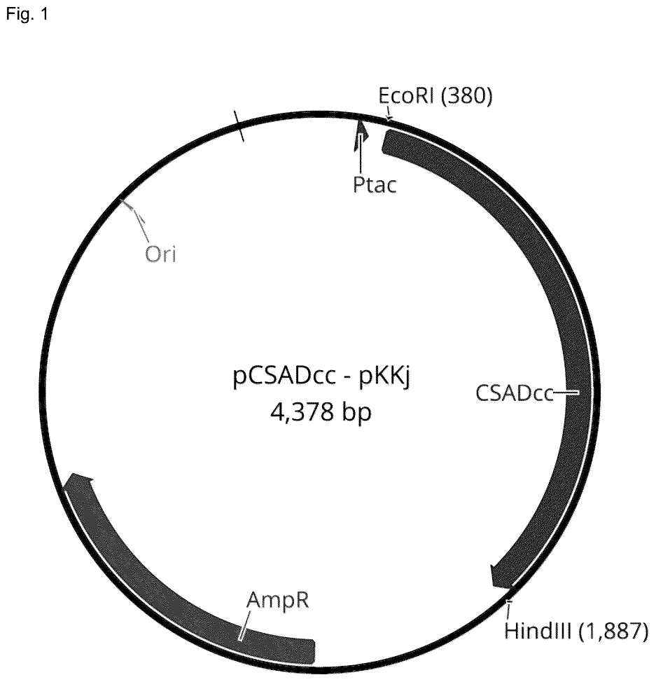

Preferably, the process is characterized in that the L-cysteine sulfinic acid decarboxylase stems from fermentative production. Corresponding recombinant production of the CSADcc enzyme in an E. coli production strain is disclosed in example 6. To this end, the CSADcc cds is cloned into an expression vector, for example the vector pKKj, in a known manner and the gene construct pCSADcc-pKKj is produced (FIG. 1). A production strain is produced by transforming the gene construct pCSADcc-pKKj into an E. coli host strain, for example the strain E. coli JM105, in a likewise known manner and using the resultant production strain E. coli JM105×pCSADcc-pKKj for production of the CSADcc enzyme in a likewise known manner. The CSADcc enzyme can be produced on a shake flask scale for laboratory purposes or by fermentation (example 6).

CSAD enzymes contain pyridoxal phosphate (PLP, CAS No. 54-47-7) as a cofactor. Supplementing the growth medium or biotransformation batches for conversion of L-cysteic acid to taurine with PLP therefore offers one way of achieving process improvement. Since PLP belongs to the vitamin B6 family, supplementation with other members of the vitamin B6 family, such as pyridoxine (CAS No. 65-23-6), pyridoxal (CAS No. 66-72-8) or pyridoxamine (CAS No. 85-87-0), is a suitable alternative for process improvement. Preferably, the biotransformation process for conversion of L-cysteic acid to taurine is characterized in that it is carried out in the presence of 20 mg/L, particularly preferably 10 mg/L and especially preferably 4 mg/L PLP.

In the process according to the invention, the CSAD enzyme, preferably CSADcc, obtained by growth in a shake flask or by fermentation, can be used either as a fermenter broth without further workup or as a cell suspension after reisolation of the cells by, for example, centrifugation and resuspension of the fermenter cells, for example in a buffer (resuspended fermenter cells). Furthermore, the CSAD enzyme, preferably CSADcc, can be used in the form of a cell homogenate after mechanical disruption of the resuspended fermenter cells or in the form of chemically permeabilized cells (e.g., by chloroform) or else as a cell extract after removal of particulate constituents from the cell homogenate or else as an enzyme purified by chromatography, for example.

Preference is given to the use of the CSAD enzyme (CSADcc) as a fermenter broth without further workup, as resuspended fermenter cells or else as a cell homogenate after mechanical disruption of the resuspended fermenter cells. Particular preference is given to the use of the CSAD enzyme as resuspended fermenter cells or as a cell homogenate after mechanical disruption of the resuspended fermenter cells. Especial preference is given to the use of the CSAD enzyme in the form of resuspended fermenter cells.

The biotransformation of L-cysteic acid to taurine by the CSAD enzyme, preferably CSADcc, is preferably carried out under pH and temperature conditions that allow efficient decarboxylation of L-cysteic acid to taurine.

The preferred pH range in which biotransformation 2 is carried out is between pH 5.0 and 9.0, particularly preferably between pH 6.0 and 8.5 and especially preferably between pH 6.5 and 8.0.

Biotransformation 2 is preferably carried out at a temperature of <70° C., preferably <60° C., particularly preferably <50° C. and especially preferably <40° C.

The molar yield of taurine from the biotransformation of L-cysteic acid in biotransformation 2 is preferably at least 60%, particularly preferably at least 80% and especially preferably at least 90%.

The biotransformation for production of taurine from L-cysteic acid can be carried out in discontinuous or continuous operation. In discontinuous operation (batch operation), all reactants are added to the batch in the course of the reaction and the batch is worked up after the reaction has ended. In continuous operation, the CSAD enzyme is charged as a stationary phase, for example immobilized in a membrane reactor or on a support, and the substrate L-cysteic acid is metered in as a mobile phase. The contact time of the mobile phase with the stationary phase is set such that the substrate L-cysteic acid can completely react to form the product taurine. Preference is given to discontinuous (batch) operation.

The preferred process for producing taurine from OAS, comprising biotransformation 1 and biotransformation 2, is characterized in that it comprises the following steps:
a) OAS is produced by fermentation,
b) the enzymes from the class of OAS sulfhydrylases (EC 4.2.99.8), such as CysM, and the class of cysteine sulfinic acid decarboxylases (EC 4.1.1.29), such as CSADcc, are produced by fermentation;
c) OAS and sulfurous acid, or a salt of sulfurous acid, react to form L-cysteic acid under enzymatic catalysis by the OAS sulfhydrylase from point b, and
d) L-cysteic acid from point c is decarboxylated to taurine by the CSAD enzyme from point b.

Especially preferably, the process comprising steps a, b, c and d is characterized in that, in biotransformation 1, OAS is reacted with Na₂SO₃, or the salt of its anhydride Na₂S₂O₅, to form L-cysteic acid in a CysM-catalyzed reaction and, in biotransformation 2, the L-cysteic acid from biotransformation 1 is decarboxylated to taurine in a CSAD-catalyzed biotransformation according to equation (5). These process steps are disclosed in examples 7 to 10. For this purpose, OAS of biotransformation 1 can be produced synthetically or by growth of a production strain, as described in example 1 of the present invention. Preference is given to producing OAS by growth of a production strain. The CysM enzyme of biotransformation 1 can stem from the fermentation of a production strain, as described in example 2. The CSAD enzyme used in biotransformation 2 can stem from the growth of a production strain, preferably the CSADcc enzyme from the growth of the strain E. coli JM105×pCSADcc-pKKj, as described in example 6. L-cysteic acid from biotransformation 1 can be used in biotransformation 2 without further workup, as described in examples 8 to 10, or else after prior workup. A person skilled in the art is familiar with various methods for this purpose, such as filtration, centrifugation, extraction, adsorption, ion exchange chromatography, precipitation, crystallization. Preference is given to the use of L-cysteic acid from biotransformation 1 in biotransformation 2 without further workup.

The process for producing taurine combines in a simple and efficient manner the hitherto undescribed enzymatic production of L-cysteic acid from OAS and a salt of sulfurous acid, also referred to as sulfite, according to equation (7) (biotransformation 1) with the enzymatic decarboxylation of L-cysteic acid to taurine according to equation (5) (biotransformation 2). This means that the present invention comprises a two-stage biotransformation process from the combination of biotransformation 1 with biotransformation 2 for producing taurine from OAS.

Preferably, the process steps (biotransformation 1 and biotransformation 2) for production of taurine proceed sequentially, i.e., one after the other. If the process comprises steps a-d in the preferred embodiment already described, it is characterized, in an alternatively preferred embodiment, in that all the process steps take place in one reaction batch. If all the process steps take place in one reaction batch, the process is also called a one-pot process or one-pot reaction. This has the advantage that all the reactants for taurine production are already present in the reaction batch or can be easily metered in. Carrying out the process in one reaction batch is of particular interest when considering economic viability.

For instance, example 11 of the present invention discloses that the process steps can take place in one batch (one-pot process), with OAS being reacted with a sulfite (salt of sulfurous acid) in the presence of the enzymes CysM and CSADcc. L-cysteic acid is formed in the first reaction according to equation (7) and it is decarboxylated “in situ” to taurine by CSADcc according to equation (5).

The product distribution of L-cysteic acid and taurine from the reaction of OAS in the one-pot process is determined by the activity of CysM in relation to CSADcc. With sufficient metered addition of CSADcc, the L-cysteic acid formed from OAS can be converted quantitatively to taurine. Preference is given to a process in which the OAS used is converted to L-cysteic acid and taurine. The total molar yield of L-cysteic acid and taurine, based on the molar amount of OAS used, is preferably more than 60%, particularly preferably more than 70% and especially preferably more than 80%. Based on the molar amount of OAS used, the molar yield of taurine is preferably more than 25%, particularly preferably more than 50% and especially preferably more than 80%.

With regard to a one-pot process, it is conceivable that the genes for the OAS sulfhydrylase and the L-cysteine sulfinic acid decarboxylase can be jointly expressed in one strain and the cells from the growth of said strain are reacted with OAS in the presence of a sulfite in a biotransformation to produce taurine as the end product.

In one modification of the invention, it is also conceivable that, in the context of a metabolic engineering approach, the genes for an OAS sulfhydrylase and an L-cysteine sulfinic acid decarboxylase are expressed in the OAS production strain and taurine is produced by growth of such a production strain in the presence of a sulfite (salt of sulfurous acid).

The FIGURE shows the plasmid used in the examples.

FIG. 1: pCSADcc-pKKj

### ABBREVIATIONS USED IN THE FIGURE

AmpR: Gene conferring resistance to ampicillin (β-lactamase)
Ori: Origin of replication
Ptac: tac promoter
EcoRI: Cleavage site for the restriction enzyme EcoRI
HindIII: Cleavage site for the restriction enzyme HindIII
CSADcc: CSAD (cysteine sulfinic acid decarboxylase) C. carpio cds

The invention will be further illustrated by the following examples without being restricted by them:

### Example 1: Production of OAS

The strain E. coli W3110/pACYC-cysEX-GAPDH-ORF306 disclosed in EP 1 233 067 B1 (Wacker) and deposited according to the Budapest Treaty at the DSMZ-German Collection of Microorganisms and Cell Cultures GmbH (Braunschweig) under the number DSM 13495 was used. OAS was produced by fermentation as described in EP 1 233 067 B1. At the end of fermentation, OAS was stabilized by setting a pH of 4.5 using 21% (v/v) phosphoric acid. The cells were removed by centrifugation at 4000 rpm for 10 min (Heraeus Megafuge 1.0 R). The HPLC-determined content of OAS in the fermentation supernatant was 15.3 g/L.

### HPLC Analysis of OAS, L-Cysteic Acid and Taurine:

For quantitative determination of the compounds analyzed in the examples, an HPLC method calibrated respectively for OAS, L-cysteic acid and taurine was employed; all reference substances used for calibration were commercially available (Sigma-Aldrich). An Agilent 1260 Infinity II HPLC system was used, which was equipped with a unit from the same manufacturer for pre-column derivatization with o-phthaldialdehyde (OPA derivatization) as is known from the analysis of amino acids. For detection of the OPA-derivatized products OAS, L-cysteic acid and taurine, the HPLC system was equipped with a fluorescence detector. The detector was set to an excitation wavelength of 330 nm and an emission wavelength of 450 nm. Also used were an Accucore™ aQ column from Thermo Scientific™, length 100 mM, internal diameter 4.6 mm, particle size 2.6 μm, thermally equilibrated at 40° C. in a column oven.

Eluent A: 25 mM Na phosphate, pH 6.0. Eluent B: methanol. The separation was carried out in gradient mode: 10% eluent B to 60% eluent B over 0-25 min, followed by 60% eluent B to 100% eluent B over 2 min, followed by 100% eluent B for a further 2 min, at a flow rate of 0.5 ml/min. Retention time of L-cysteic acid: 3.2 min. Retention time of taurine: 14.8 min. Retention time of OAS: 17.0 min.

### Example 2: Production of the Enzyme CysM

The strain E. coli DH5α/pFL145 disclosed in EP 1 247 869 B1 (Wacker) and deposited according to the Budapest Treaty at the DSMZ-German Collection of Microorganisms and Cell Cultures GmbH (Braunschweig) under the number DSM 14088 was used. CysM enzyme was produced both by growth in a shake flask and by fermentation.
A) Growth in a shake flask: A preculture of the strain E. coli DH5α/pFL145 was prepared in Lbamp medium (10 g/l tryptone (GIBCO™), 5 g/l yeast extract (BD Biosciences), 5 g/l NaCl, 100 mg/L ampicillin (Sigma-Aldrich)) (growth at 37° C. and 120 rpm overnight). 25 ml of preculture were used as inoculum for a main culture of 250 ml of LBamp medium (1 L Erlenmeyer flask with baffles). The main culture was shaken at 30° C. and 110 rpm. After 4 h, a cell density OD₆₀₀ of 1.0/ml was reached (OD₆₀₀: photometric determination of cell density per ml of cell suspension by determination of absorbance at 600 nm; Genesys™ 10S UV-Vis spectrophotometer from Thermo Scientific™). Then the inducer tetracycline (Sigma-Aldrich, 3 mg/L final concentration) was added and growth was continued for another 20 h at 30° C. and 110 rpm. At the end of growth, the cell density OD₆₀₀ was 3/ml.
B) Fermentative production of CysM with the strain E. coli DH5α/pFL145 is disclosed in EP 1 247 869 B1. The cells from the fermentation were removed by centrifugation at 4000 rpm for 10 min (Heraeus Megafuge 1.0 R) and suspended in KPi6.5 buffer (0.1 M K phosphate, pH 6.5), so that the cell density OD₆₀₀ was 90/ml.

The cells from the shake flask growth or fermentation were isolated by centrifugation (10 min at 15000 rpm, Sorvall RC5C centrifuge, equipped with an SS34 rotor) for further use. For the further use for production of a cell homogenate, as described below, the cell pellet was resuspended in KPi6.5 buffer as a cell suspension. The cell suspension was prepared by using an amount of KPi6.5 buffer sufficient for the cell density OD₆₀₀ to be 30/ml: for example, 50 ml of cells from the shake flask growth having an OD₆₀₀ of 3/ml were centrifuged and resuspended in 5 ml of KPi6.5 buffer (10-fold concentration) or 1 ml of cells from the fermentation having an OD₆₀₀ of 90/ml were resuspended in 3 ml of KPi6.5 buffer (3-fold dilution).

This produced the cells of the strain E. coli DH5α/pFL145, isolated from the fermenter broth and resuspended, that were used in the following as OAS sulfhydrylase CysM in the process according to the invention.

To prepare a cell homogenate, the FastPrep-24™ 5G cell homogenizer from MP Biomedicals was used. 1 ml of cell suspension in KPi6.5 buffer having a cell density OD₆₀₀ of 30/ml was disrupted in manufacturer-assembled 1.5 ml tubes containing glass beads (“Lysing Matrix B”) (3×20 sec at a shaking frequency of 6000 rpm with a 30 sec pause each time between the intervals). The cell homogenate obtained was used directly as OAS sulfhydrylase (CysM enzyme) in the process according to the invention or used for preparation of a cell extract.

To prepare a cell extract, the cell homogenate obtained was centrifuged (15 000 rpm for 10 min, Sorvall RC5C centrifuge, equipped with an SS34 rotor), and the supernatant designated cell extract and used as OAS sulfhydrylase (CysM enzyme) in the process according to the invention or used further for determination of CysM enzyme activity.

The protein content of the cell extract was determined by means of a Qubit 3.0 Fluorometer from Thermo Fisher Scientific using the “Qubit© Protein Assay Kit” according to the manufacturer's instructions. The protein content of the cell extract from the shake flask growth was 5.3 mg/ml. The protein content of the cell extract from the fermentation was 4.0 mg/ml.

CysM enzyme activity was determined as described in EP 1 247 869 B1 (Wacker). To this end, OAS (Sigma-Aldrich) was incubated at 37° C. in the presence of Na₂S and cell extract from the growth of the strain E. coli DH5α/pFL145. The assay (0.4 ml final volume) in KPi6.5 buffer contained 10 mM OAS (addition from a 200 mM stock solution in 500 mM sodium succinate buffer pH 5.5), 10 mM sodium sulfide Na₂S and 5 μl of CysM-containing cell extract. The cysteine produced in the CysM reaction was determined using ninhydrin (Sigma-Aldrich) according to the method by Gaitonde (1967), Biochem. J. 104: 627-633. The CysM enzyme activity in the cell extract from the growth of the strain E. coli DH5α/pFL145 in a shake flask was 57.1 U/ml. Since the cells from the shake flask growth (OD₀₀₀ of 3/ml) had been concentrated 10-fold for the preparation of the cell extract, the enzyme activity in cells from the shake flask growth was 5.7 U/ml. The CysM enzyme activity in the cell extract following fermentation of the strain E. coli DH5α/pFL145 was 58.1 U/ml. Since the cells from the fermentation (OD₆₀₀ of 90/ml) had been diluted to an OD₆₀₀ of 30/ml for preparation of the cell extract, the enzyme activity in the concentrated (OD₀₀₀ of 90/ml) cell suspension of the fermenter cells was 174.4 U/ml.

The specific CysM enzyme activity of the cell extract from the growth of the strain E. coli DH5α/pFL145 in a shake flask was 10.8 U per mg of protein. The specific CysM enzyme activity of the cell extract following fermentation of the strain E. coli DH5α/pFL145 was 14.5 U/mg. Assuming that CysM activity was completely released from the cells during the preparation of the cell extract, the CysM enzyme activity determined in the cell extracts was equated to the enzyme activity present in CysM cell suspensions in the following examples.

1 U/ml CysM enzyme activity is defined as the production of 1 μmol of cysteine per min from OAS and Na₂S under assay conditions in 1 ml of cell extract (volume activity). Specific CysM enzyme activity in U per mg of protein is obtained by dividing the volume activity of the cell extract (U/ml) by the protein concentration of the cell extract (mg/ml) and is defined as CysM enzyme activity in U based on 1 mg of protein in the cell extract.

### Example 3: Production of L-Cysteic Acid from Commercially Available OAS and Na₂SO₃ with the Aid of CysM Produced in a Shake Flask Culture

Two batches were carried out in parallel:

Batch 1: A 100 ml Erlenmeyer flask was initially charged with 8.25 ml of NaPi6.5 buffer (50 mM Na phosphate, pH 6.5), and added in succession were 1 ml of a 0.2 M solution of Na₂SO₃ in NaPi6.5 buffer, 0.4 ml of CysM cell extract from the shake flask growth (from example 2A) having an activity of 57.1 U/ml (2.3 U/ml final concentration in the batch) and 350 μl of a 0.2 M solution of OAS×HCl (Sigma-Aldrich) in 0.5 M Na succinate, pH 5.5. The batch volume was 10 ml.

Batch 2: The batch (comparative batch without Na₂SO₃) had the same composition as batch 1. Instead of the Na₂SO₃ solution, batch 2 received 1 ml of NaPi6.5 buffer.

Both batches were incubated at 37° C. and 140 rpm in a chest shaker (Infors). After 1 h and 3 h, 1 ml of the batches in each case were incubated at 80° C. for 5 min to stop the reaction and centrifuged, and the supernatant was analyzed by HPLC. The amount of L-cysteic acid detected by HPLC is shown in Table 1.

TABLE 1
HPLC-detected amount of L-cysteic acid according to
reaction time, using commercially available OAS and Na₂SO₃
and a CysM-containing cell extract.

| | Batch 1 with Na₂SO₃ | Batch 2 without Na₂SO₃ |
|---|---|---|
| Time [h] | L-cysteic acid [mg/L] | L-cysteic acid [mg/L] |
| 0 | 0.0 | 0.0 |
| 1 | 78.0 | 0.0 |
| 3 | 95.8 | 0.0 |

### Example 4: Production of L-Cysteic Acid from OAS-Containing Culture Supernatant from Fermentation and Na₂SO₃ with the Aid of CysM Produced in a Shake Flask Culture

A 100 ml Erlenmeyer flask was initially charged with 1 ml of cell culture supernatant from the fermentation of the strain E. coli W3110/pACYC-cysEX-GAPDH-ORF306 having an OAS content of 15.3 g/L (from example 1), and added in succession were 6 ml of NaPi6.5 buffer, 1 ml of a 1 M solution of Na₂SO₃ in NaPi6.5 buffer and 2 ml of CysM cell suspension from the shake flask growth (from example 2A, cell density OD₆₀₀ of 30/ml; 57.1 U/ml of CysM enzyme activity). The batch volume was 10 ml. The CysM enzyme activity in the batch was 11.4 U/ml. The batch was incubated at 37° C. and 140 rpm in a chest shaker (Infors). After 2 h, 1 ml of the batch was incubated at 80° C. for 5 min and centrifuged and the supernatant was analyzed by HPLC for the content of OAS and L-cysteic acid. The course of the reaction over time is summarized in Table 2.

TABLE 2
HPLC-detected amount of L-cysteic acid and OAS,
using an OAS-containing cell culture supernatant, Na₂SO₃
and a CysM-containing cell suspension.

| Time [h] | OAS [mg/L] | L-cysteic acid [mg/L] |
|---|---|---|
| 0 | 1530.0 | 0.0 |
| 2 | 0.0 | 1473.3 |

### Example 5: Preparative Production of L-Cysteic Acid by Biotransformation of OAS at Constant pH

A 0.5 L thermostatable double-walled glass vessel (Diehm) was connected to a thermostat (Lauda) via a hose connection and adjusted to a temperature of 37° C. 50 ml of CysM-containing cell suspension in KPi6.5 buffer (OD₆₀₀ of 90/ml, 8720 U of CysM enzyme activity) from the fermentation of the strain DH5α/pFL145 (from example 2B) and 6.6 ml of a 400 g/L solution of Na₂S₂O₅ (13.9 mmol, molecular weight of 190.1 g/mol) in KPi6.5 buffer were initially charged. In dissolved form, this corresponded to 27.8 mmol of NaHSO3 (1.78-fold molar excess to the OAS amount of 15.6 mmol that is metered in later). The batch was stirred with a magnetic stirrer. The batch was also equipped with a pH electrode (Mettler Toledo), which was connected to a pH control unit (TitroLine alpha titrator, Schott) which was operated in pH-stat mode according to the manufacturer's instructions. Under pH-stat conditions, the pH in the reaction vessel was kept constant at the set pH of 6.5 over the entire duration of the reaction by metered addition of 2 M NaOH from a burette connected to the control unit. 150 ml of OAS-containing cell culture supernatant (OAS content: 15.3 g/L, 2.3 g; 15.64 mmol) from the fermentation of the strain E. coli W3110/pACYC-cysEX-GAPDH-ORF306 (example 1) were metered into the batch from a reservoir via a pump (Watson Marlow 101 U/R peristaltic pump) at a flow rate of 0.35 ml/min.

The reaction time was 19 h. Since the batch was carried out in an open reaction vessel, the batch volume was 185 ml after completion of the reaction owing to evaporation. 0.5 h, 3 h and 19 h after the start of the reaction, a 1 ml aliquot of the batch was removed in each case and the content of L-cysteic acid was analyzed by HPLC. The formation of L-cysteic acid over time is summarized in Table 3. After a 19 h reaction time, the L-cysteic acid content in the batch was 12 970 mg/L (76.65 mM), which corresponded to an absolute molar yield of 14.18 mmol of L-cysteic acid for a batch volume of 185 ml. Based on the amount of OAS used of 15.64 mmol, this corresponded to a yield of 90.1%.

TABLE 3
HPLC-detected amount of L-cysteic acid according to reaction
time, using an OAS-containing fermentation supernatant, NaHSO₃
and a cell suspension of CysM-containing fermenter cells

| Time [h] | L-cysteic acid [mg/L] | L-cysteic acid [mM] |
|---|---|---|
| 0.5 | 758.0 | 4.47 |
| 3 | 4244.0 | 25.08 |
| 19 | 12970.0 | 76.65 |

### Example 6: Recombinant Production of CSADcc from Cyprinus carpio (Carp) in E. coli

Vector pCSADcc-pKKj:

The cDNA gene (cDNA: complementary DNA, isolated from mRNA by reverse transcription) of the cysteine sulfinic acid decarboxylase (CSAD) from Cyprinus carpio (carp) was isolated by Honjoh et al. (2010), Amino Acids 38: 1173-1183 and the DNA sequence disclosed in the NCBI (National Center for Biotechnology Information) database under the GenBank sequence ID: AB220585.1 (cds: nt 82-1584). The corresponding amino acid sequence was used to derive a DNA sequence codon-optimized for expression in E. coli (publicly available Eurofins Genomics GENEius software), which was synthetically produced (Eurofins Genomics). The synthetically produced DNA had the sequence disclosed in SEQ ID NO: 1 and contained the cds of the gene, referred to hereinafter as CSADcc cds (SEQ ID NO: 1, nt 31-1530), encoding a protein having the amino acid sequence disclosed in SEQ ID NO: 2 and referred to as CSADcc. For cloning purposes, the synthetically produced DNA contained at the 5′ end an EcoRI cleavage site (SEQ ID NO: 1, nt 25 to 30) and at the 3′ end a HindIII cleavage site (SEQ ID NO: 1, nt 1532 to 1537).

The vector pCSADcc-pKKj suitable for recombinant expression of the CSADcc cds (FIG. 1) was produced by cutting the synthetically produced DNA using EcoRI and HindIII and cloning it in a known manner as an EcoRI/HindIII fragment into the vector pKKj cut using EcoRI and HindIII. The expression vector pKKj, disclosed in EP 2 670 837 A1 (Wacker), is a derivative of the expression vector pKK223-3. The DNA sequence of pKK223-3 is disclosed in the GenBank gene database under the accession number M77749.1. From the 4.6 kb plasmid, approx. 1.7 kb (bp 262-1947 of the DNA sequence disclosed in M77749.1) were removed, thereby yielding the 2.9 kb expression vector pKKj.

E. coli JM105×pCSADcc-pKKj Production Strain:

The CSADcc cds was expressed in E. coli by transforming the vector pCSADcc-pKKj in a known manner into the strain E. coli K12 JM105. The strain E. coli K12 JM105 is commercially available under the strain number DSM 3949 from the DSMZ-German Collection of Microorganisms and Cell Cultures GmbH.

Clones from the transformation were selected on LBamp plates. LBamp plates contained 10 g/l tryptone (GIBCO™), 5 g/l yeast extract (BD Biosciences), 5 g/l NaCl, 15 g/l agar and 100 mg/L ampicillin (Sigma-Aldrich). A clone was selected for shake flask growth and fermentation. The CSADcc-producing strain was designated E. coli JM105×pCSADcc-pKKj. The CSADcc cds was expressed in E. coli JM105×pCSADcc-pKKj in a known manner under the control of the IPTG-inducible tac promoter (IPTG: isopropyl β-thiogalactoside, Sigma-Aldrich) functionally linked to the CSADcc cds.

### Growth in a Shake Flask:

A preculture of the strain E. coli JM105×pCSADcc-pKKj was prepared in LBamp medium (growth at 37° C. and 120 rpm overnight, Infors chest shaker).

2 ml of preculture were used as inoculum for a main culture of 100 ml of SM3 medium (1 L Erlenmeyer flask), supplemented with 15 g/L glucose, 5 mg/L pyridoxal phosphate (PLP, Sigma-Aldrich) and 100 mg/L ampicillin. The main culture was shaken at 30° C. and 140 rpm in a chest shaker (Infors). After a 4 h incubation time, a cell density OD₆₀₀ of 2.0 was reached. Then the inducer IPTG (Sigma-Aldrich, 0.4 mM final concentration) was added and growth was continued for another 20 h at 30° C. and 140 rpm in a chest shaker (Infors).

Composition of the SM3 medium: 12 g/L K₂HPO₄, 3 g/L KH₂PO₄, 5 g/L (NH₄)₂SO₄, 0.3 g/L MgSO₄×7 H₂O, 0.015 g/L CaCl₂)×2H₂O, 0.002 g/L FeSO₄×7 H₂O, 1 g/L Na₃ citrate×2H₂O, 0.1 g/L NaCl; 5 g/L peptone (Oxoid); 2.5 g/L yeast extract (BD Biosciences); 0.005 g/L vitamin B1; 1 ml/L trace element solution.

Composition of the trace element solution: 0.15 g/L of Na₂MoO₄·2H₂O, 2.5 g/L of H₃BO₃, 0.7 g/L of COCl₂·6H₂O, 0.25 g/L of CuSO₄·5H₂O, 1.6 g/L of MnCl₂·4H₂O, 0.3 g/L of ZnSO₄·7H₂O.

The cells from the shake flask growth were isolated by centrifugation. A cell suspension was prepared by resuspending the cell pellet from 50 ml of the shake flask growth in 2 ml of 50 mm Na phosphate, pH 7.0 (NaPi7.0 buffer). The cell suspension was either used directly for biotransformation tests or used for preparation of a cell homogenate.

To prepare a cell homogenate, the FastPrep-24™ 5G cell homogenizer from MP Biomedicals was used. 2×1 ml of cell suspension were disrupted in manufacturer-assembled 1.5 ml tubes containing glass beads (“Lysing Matrix B”) (3×20 sec at a shaking frequency of 6000 rpm with a 30 sec pause each time between the intervals).

The cell homogenate obtained (2 ml volume) was used without further workup for the biotransformation of L-cysteic acid to taurine.

### Growth by Fermentation:

The production strain E. coli JM105×pCSADcc-pKKj was used for the fermentation. The fermentation was carried out in a Biostat B fermenter (2 L working volume) from Sartorius BBI Systems GmbH.

### Shake Flask Preculture:

100 ml of LBamp medium in a 1 L Erlenmeyer flask was inoculated from an agar plate containing the strain JM105×pCSADcc-pKKj and incubated on an incubation shaker (Infors) at 30° C. and a speed of 120 rpm for 7-8 h up to a cell density of OD₆₀₀ of 2/ml-4/ml.

### Prefermenter:

1.5 L of FM2 medium, supplemented with 40 g/L glucose and 100 mg/L ampicillin, were inoculated with 21.3 ml of the shake flask preculture. The fermentation conditions were: temperature of 30° C.; constant pH of pH 7.0 (automatic correction with 25% NH₄OH and 6.8 N H₃PO₄); foam control by automatic metered addition of 4% v/v Struktol J673 in H₂O (Schill & Seilacher); stirrer speed of 450-1300 rpm; constant aeration at 1.7 vvm with compressed air sterilized by a sterile filter (vvm: introduction of compressed air into the fermentation batch expressed in liters of compressed air per liter of fermentation volume per minute); pO₂≥50%. The partial pressure of oxygen pO₂ was regulated via the stirring speed. After a fermentation time of 16 h, a cell density OD₆₀₀ of 45/ml was reached.

### Production Fermenter:

1.35 L of FM2 medium, pH 7.0, supplemented with 20 g/L glucose, 0.36 g/L pyridoxine (vitamin B6, Sigma-Aldrich) and 100 mg/L ampicillin, were inoculated with 150 ml of prefermenter culture. The fermentation conditions were: temperature of 30° C.; constant pH of pH 7.0 (automatic correction with 25% NH₄OH and 6.8 N H₃PO₄); foam control by automatic metered addition of 4% v/v Struktol J673 in H₂O (Schill & Seilacher); stirrer speed of 450-1300 rpm; constant aeration at 1.7 vvm; pO₂≥50%. The partial pressure of oxygen pO₂ was regulated via the stirring speed. The fermentation time was 30 h.

FM2 medium: (NH₄)₂SO₄, 5 g/L; NaCl, 0.50 g/L; FeSO₄×7 H₂O, 0.075 g/L; Nas citrate, 1 g/L; MgSO₄×7 H₂O, 0.30 g/L; CaCl₂×2 H₂O, 0.015 g/L; KH₂PO₄, 1.50 g/L; vitamin B1 (Sigma-Aldrich), 0.005 g/L; peptone (Oxoid), 5.00 g/L; yeast extract (Oxoid), 2.50 g/L; trace element solution, 10 ml/L (corresponds to the one used for the shake flask growth).

The pH in the fermenter was initially adjusted to 7.0 by pumping in a 25% NH₄OH solution. During the fermentation, the pH was maintained at a value of 7.0 by automatic correction with 25% NH₄OH or 6.8 N H₃PO₄. For inoculation, 150 ml of the prefermenter culture were pumped into the fermenter vessel. The initial volume was thus 1.5 L. The cultures were initially stirred at 350 rpm and aerated at an aeration rate of 1.7 vvm. Under these starting conditions, the oxygen probe was calibrated to 100% saturation prior to inoculation.

The target value for the O₂ saturation (pO₂) during the fermentation was set to 50%. After the O₂ saturation had fallen below the target value, a regulation cascade was started in order to bring the O₂ saturation back up to the target value. In this connection, the stirring speed was continuously increased (up to a maximum of 1300 rpm).

The fermentation was carried out at a temperature of 30° C. Once the glucose content in the fermenter had fallen from an initial 20 g/L to approx. 5 g/L, a 60% (w/w) glucose solution was continuously metered in. The feeding rate was adjusted such that the glucose concentration in the fermenter no longer exceeded 2 g/L from then on. Glucose was determined using a glucose analyzer from YSI (Yellow Springs, Ohio, USA).

Once the cell density in the fermenter had reached an OD₆₀₀ of 50/ml (fermentation time of 8 h), the expression of the CSADcc gene was started by a single addition of the inducer IPTG (final concentration of 0.2 mm). 22 h after induction, corresponding to a total fermentation time of 30 h, the fermentation was stopped. At this moment, the cell density OD₆₀₀ was 164/ml. 1 L of the fermenter broth was centrifuged (15 000 rpm for 10 min, Sorvall RC5C centrifuge, equipped with an SS34 rotor), the fermentation supernatant was discarded, and the cells were resuspended in 1 L of NaPi7.0 buffer and stored in 50 ml aliquots at −20° C. for further use.

### Example 7: Production of Taurine from Commercially Available L-Cysteic Acid by Biotransformation

12 mg of L-cysteic acid×H₂O (Sigma-Aldrich) were weighed in a 100 ml Erlenmeyer flask and dissolved in 9.7 ml of NaPi7.0 buffer. The reaction was started by addition of 0.3 ml of cell homogenate from the shake flask growth of JM105×pCSADcc-pKKj (example 6). The batch volume was 10 ml. The molar concentration of L-cysteic acid×H₂O was 6.41 mM (molecular weight of L-cysteic acid×H₂O: 187.2 g/mol). The batch was incubated at 37° C. and 140 rpm in a chest shaker (Infors). After 3 h, 1 ml of the batch was incubated at 80° C. for 5 min and centrifuged and the supernatant was analyzed by HPLC. The L-cysteic acid used was completely consumed. The amount of taurine formed was 789.4 mg/L, corresponding to a molar content of 6.31 mM (molecular weight of taurine: 125.1 g/mol). Accordingly, the molar yield of taurine formed from 6.41 mM L-cysteic acid×H₂O was 98.4%.

### Example 8: Production of Taurine from Commercially Available OAS by Biotransformation

Reaction 1: Production of L-Cysteic Acid from OAS:

A 100 ml Erlenmeyer flask was initially charged with 6.6 ml of KPi6.5 buffer, and added in quick succession were 0.4 ml of a 0.2 M stock solution of OAS×HCl (Sigma-Aldrich), dissolved in 0.5 M Na succinate, pH 5.5, 1 ml of 1 M Na₂SO₃ in KPi6.5 buffer, and 2 ml of cell suspension of cysM cells from the shake flask growth (from example 2A; CysM enzyme activity of 57.1 U/ml). The batch volume was 10 ml. The metered amount of CysM enzyme in the batch was 11.4 U/ml). The molar concentration of OAS×HCl was 8.00 mM (1.47 g/L; molecular weight of OAS×HCl: 183.6 g/mol). The batch was incubated at 37° C. and 140 rpm in a chest shaker (Infors). After 3 h, 1 ml of the batch was incubated at 80° C. for 5 min and centrifuged and the supernatant was analyzed by HPLC. The OAS used was completely consumed. The amount of L-cysteic acid formed was 1350.4 mg/L, corresponding to a molar content of 7.98 mM (molecular weight of L-cysteic acid: 169.2 g/mol). The molar yield of L-cysteic acid formed from 8.00 mM OAS×HCl was 99.7%.

Reaction 2: Production of taurine from the L-cysteic acid synthesized in reaction 1: A 100 ml Erlenmeyer flask was initially charged with 9 ml of the batch from reaction 1, the pH was adjusted to pH 7.0 with 1 M KOH, and 1 ml of cell homogenate from the shake flask growth of the strain JM105×pCSADcc-pKKj was added (example 6). The batch volume was 10 ml. On the basis of an L-cysteic acid content of 1350.4 mg/L from reaction 1, the L-cysteic acid content at the start of reaction 2 was 1215.4 mg/L (7.18 mM at a molecular weight of 169.2 for L-cysteic acid). The batch was incubated at 37° C. and 140 rpm in a chest shaker (Infors). After 3 h, 1 ml of the batch was incubated at 80° C. for 5 min and centrifuged and the supernatant was analyzed by HPLC. The L-cysteic acid used was completely consumed. The content of taurine was 825.7 mg/L (6.60 mM at a molecular weight for taurine of 125.1 g/mol). The molar yield of taurine formed from 7.18 mM L-cysteic acid was 91.9%.

### Example 9: Production of Taurine by CSADcc-Catalyzed Conversion of L-Cysteic Acid from the Reaction of Fermentatively Produced OAS with NaHSO₃ and Fermentatively Produced CysM Enzyme

In a 100 ml Erlenmeyer flask, 7 ml of NaPi7.0 buffer containing 5 mg/L PLP, 1 ml of the batch from example 5 having a content of L-cysteic acid of 12 970 mg/L and 2 ml of CSADcc cells in NaPi7.0 buffer (example 6) were mixed. The batch volume was 10 ml. The L-cysteic acid content in the batch was 1297 mg/L, corresponding to a molar content of 7.67 mM (molecular weight of L-cysteic acid: 169.2 g/mol). The batch was incubated at 37° C. and 140 rpm in a chest shaker (Infors). After 4 h, 1 ml of the batch was incubated at 80° C. for 5 min and centrifuged and the supernatant was analyzed by HPLC. The L-cysteic acid used was completely consumed. The amount of taurine formed was 925.3 mg/L, corresponding to a molar content of 7.40 mM (molecular weight of taurine: 125.1 g/mol). The molar yield of taurine formed from 7.67 mM L-cysteic acid was 96.4%.

### Example 10: Preparative Production of Taurine

### Biotransformation 1:

A 0.5 L thermostatable double-walled glass vessel (Diehm) was connected to a thermostat (Lauda) via a hose connection and adjusted to a temperature of 37° C. 50 ml of CysM-containing cell suspension in KPi6.5 buffer (OD₆₀₀ of 90/ml, 8720 U of CysM enzyme activity) from the fermentation of the strain DH5α/pFL145 (from example 2) and 20 ml of a 400 g/L solution of Na₂S₂O₅ (42.1 mmol, molecular weight of 190.1 g/mol) in KPi6.5 buffer were initially charged. In dissolved form, this corresponded to 84.2 mmol of NaHSO3 (1.63-fold molar excess to the OAS amount of 51.7 mmol that is metered in later). The batch was stirred with a magnetic stirrer. The batch was also equipped with a pH electrode (Mettler Toledo), which was connected to a pH control unit (TitroLine alpha titrator, Schott) which was operated in pH-stat mode according to the manufacturer's instructions. Under pH-stat conditions, the pH in the reaction vessel was kept constant at the set pH of 6.5 over the entire duration of the reaction by metered addition of 2 M NaOH from a burette connected to the control unit. 400 ml of OAS-containing fermentation supernatant (OAS content: 19.1 g/L, 7.6 g; 51.7 mmol) from the fermentation of the strain E. coli W3110/pACYC-cysEX-GAPDH-ORF306 (example 1) were metered into the batch from a reservoir via a pump (Watson Marlow 101 U/R peristaltic pump) at a flow rate of 0.2 ml/min. The total reaction time was 46 h. The batch volume after completion of the reaction was 500 ml. 1 h, 3 h, 20 h, 28 h and 46 h after the start of the reaction, a 1 ml aliquot of the batch was removed in each case, incubated at 80° C. for 5 min and centrifuged, 5 and the supernatant was analyzed by HPLC for the content of L-cysteic acid. The formation of L-cysteic acid over time is summarized in Table 4. After a 46 h reaction time, the L-cysteic acid content in the batch was 15 370 mg/L (90.8 mM), which corresponded to an absolute molar yield of 45.4 mmol of L-cysteic acid for a batch volume of 500 ml. Based on the amount of OAS used of 51.7 mmol, this corresponded to a yield of 87.8%.

TABLE 4
HPLC-detected amount of L-cysteic acid according to reaction
time, using an OAS-containing fermentation supernatant, NaHSO₃
and a cell suspension of CysM-containing fermenter cells

| Time [h] | L-cysteic acid [mg/L] | L-cysteic acid [mM] |
|---|---|---|
| 1 | 709.0 | 4.2 |
| 3 | 1427.0 | 8.4 |
| 20 | 9814.0 | 58.0 |
| 28 | 10560.0 | 62.4 |
| 46 | 15370.0 | 90.8 |

### Biotransformation 2:

A 0.3 L thermostatable double-walled glass vessel (Diehm) was connected to a thermostat (Lauda) via a hose connection and adjusted to a temperature of 37° C. 100 ml of L-cysteic acid-containing biotransformation 1 were adjusted to pH 7.0 with 2.5 M NaOH. In addition, 1 ml of 1 M DTE (dithioerythritol, Sigma-Aldrich), dissolved in H₂O, 1 ml of 500 mg/L PLP (4 mg/L final concentration), dissolved in NaPi7.0 buffer, and 20 ml of CSADcc-containing fermenter cells resuspended in NaPi7.0 buffer were added. The batch was stirred with a magnetic stirrer. The batch volume was 122 ml. At the start of the reaction and then after 2 h, 4 h, 6 h and 24 h, a 1 ml aliquot of the batch was removed in each case, incubated at 80° C. for 5 min and centrifuged, and the supernatant was determined by HPLC for the content of L-cysteic acid and taurine. The result is summarized in Table 5. Based on the molar amount of L-cysteic acid used in the batch of 74.4 mM, a taurine yield (8.8 g/L, 70.2 mM) of 94.3% was achieved.

TABLE 5
HPLC-detected amount of L-cysteic acid and taurine according
to reaction time, using an L-cysteine-containing fermentation
supernatant from biotransformation 1 and a cell suspension
of CSADcc-containing fermenter cells

| | L-cysteic acid | L-cysteic acid | Taurine | Taurine |
|---|---|---|---|---|
| Time [h] | [mg/L] | [mM] | [mg/L] | [mM] |
| 0 | 12592.5 | 74.4 | 0.0 | 0.0 |
| 2 | 11821.1 | 69.88 | 1792.2 | 14.3 |
| 4 | 9106.7 | 53.83 | 2737.1 | 21.9 |
| 6 | 5159.6 | 30.5 | 4023.9 | 32.2 |
| 24 | 321.2 | 1.9 | 8783.6 | 70.2 |

### Example 11: Production of Taurine from OAS in a “One-Pot Reaction”

The reaction batch was composed of 3 ml of KPi6.5 buffer, 2 ml of OAS-containing fermentation supernatant from the fermentation of the strain E. coli W3110/pACYC-cysEX-GAPDH-ORF306 (example 1), 1 ml of 1 M Na₂SO₃ in KPi6.5 buffer, 2 ml of cell suspension of CysM-containing cells from the fermentation of the strain DH5α/pFL145 (example 2B) and 2 ml of cell suspension of CSADcc-containing cells from the shake flask growth of the strain JM105×pCSADcc-pKKj (example 6). The batch volume was 10 ml. The OAS concentration in the batch was 3.1 g/L (20.80 mM). The Na₂SO₃ concentration in the batch was 100 mM. The CysM enzyme activity in the batch was 34.9 U/ml.

The reaction was carried out at pH 6.5. The batch was incubated at 37° C. and 140 rpm in a chest shaker (Infors). 24 h after the start of the reaction, 1 ml of the batch was incubated at 80° C. for 5 min and centrifuged and the supernatant was analyzed by HPLC. The content of L-cysteic acid was 1.3 g/L (7.68 mM at a molecular weight for L-cysteic acid of 169.2 g/mol). The content of taurine was 721 mg/L (5.76 mM at a molecular weight for taurine of 125.1 g/mol). The molar yield of 7.68 mM L-cysteic acid formed from 20.80 mM OAS was 36.9%. The molar yield of 5.76 mM taurine formed from 20.80 mM OAS was 27.7%. Overall, the molar yield of L-cysteic acid and taurine formed from OAS was 64.6%.

1-10. (canceled)

11. A process for producing taurine from O-acetyl-L-serine (OAS) using biotransformation, wherein
i) wherein in a first process step (biotransformation 1), L-cysteic acid is produced from O-acetyl-L-serine (OAS) using an enzyme selected from a class of OAS sulfhydrylases (EC 4.2.99.8) in the presence of a salt of sulfurous acid, wherein the biotransformation is carried out under active pH control;
ii) wherein in a second process step (biotransformation 2), L-cysteic acid is decarboxylated to taurine;
wherein the OAS concentration in the batch is at least 10 g/L; and
wherein the OAS sulfhydrylase is CysM.

12. The process of claim 11, wherein the OAS sulfhydrylase is a bacterial enzyme.

13. The process of claim 11, wherein the OAS sulfhydrylase is CysM from the strain E. coli.

14. The process of claim 11, wherein the OAS sulfhydrylase stems from fermentative production.

15. The process of claim 11, wherein the concentration of the salt of sulfurous acid is at least in equimolar concentration to OAS.

16. The process of claim 11, wherein the L-cysteic acid is decarboxylated to taurine using an enzyme from the class of L-cysteine sulfinic acid decarboxylases (EC 4.1.1.29), aspartate 1-decarboxylases (EC 4.1.1.11) or glutamate decarboxylases (EC 4.1.1.15).

17. The process of claim 16, wherein the L-cysteine sulfinic acid decarboxylase is SEQ ID NO: 2 or a sequence homologous to this sequence.

18. The process of claim 16, wherein the L-cysteine sulfinic acid decarboxylase stems from fermentative production.

19. The process of claim 11,
a) wherein the OAS is produced by fermentation;
b) wherein the enzymes from the class of OAS sulfhydrylases (EC 4.2.99.8) and wherein the class of cysteine sulfinic acid decarboxylases (EC 4.1.1.29) are produced by fermentation;
c) wherein the OAS and a salt of sulfurous acid react to form L-cysteic acid under enzymatic catalysis by the OAS sulfhydrylase from point b; and
d) wherein the L-cysteic acid from point c is decarboxylated to taurine by the CSAD enzyme from point b.

20. The process of claim 19, wherein all of the process steps take place in one reaction batch.

21. The process of claim 11, wherein the molar yield of taurine from the biotransformation of L-cysteic acid in biotransformation 2 is preferably at least 60%.
